(19) United States
(12) Patent Application Publication
(10) Pub. No.: US 2002/0044962 A1
(43) Pub. Date: Apr. 18, 2002

(54) Encapsulation products for controlled or extended release

(76) Inventors: S. Rao Cherukuri, Frederick, MD (US); Vittorino Ravelli, Milano (IT)

Correspondence Address: Gary M. Nath, NATH & ASSOCIATES PLLC, 6th Floor, 1030 15th Street, Washington, DC, 20005, US

(21) Appl. No.: 09/982,092

(22) Filed: Oct. 19, 2001

Related U.S. Application Data
(63) Continuation-in-part of application No. 09/587,971, filed on Jun. 6, 2000.
(60) Provisional application No. 60/308,568, filed on Jul. 31, 2001.

(51) Int. Cl. A61K 9/56; A61K 9/60; A61K 9/62

(52) U.S. Cl. 424/459; 424/461

(57) ABSTRACT

A novel extended or controlled release encapsulated product is provided and includes: at least one active ingredient; at least one erodible polymer; and at least one lubricating material; wherein the encapsulated product is in the form of a caplet having a diameter of from about 1 millimeter to about 7 millimeters and a length from about 1 millimeter to about 7 millimeters. A method for preparing the encapsulated product is also provided.

### BACKGROUND OF THE INVENTION

1. Field of the Invention

The present invention relates to an encapsulation process, and in particular, an alternate encapsulation process for concentrating additives using compression. Also, the present inventive subject matter relates to encapsulation products that provide controlled or extended release of actives.

2. Description of the Prior Art

Various types of chewable articles are known in commerce. These articles include food items such as food items, confectionery items and chewing gum. The chewable articles often include various types of active agents or ingredients within the chewable articles. Examples of such active ingredients include flavors, sweeteners, colors, medicaments, pharmaceuticals, vitamins, minerals, and other effervescent agents.

It has been known in the art of food stuff, confectionery and chewing gum preparation to provide protection to the active ingredients by the use of protection systems, including providing a protective coating around the active ingredient or encapsulating the active ingredient. Such protective systems have been employed for various reasons, such as for protection of the active ingredient, both while on the shelf and during use, and for prolonged release in the oral cavity.

It is known in the art to protect active ingredients by encapsulating the active ingredient prior to introducing the ingredient into a final product. Some of the major classifications of encapsulation technology include liquid suspending media (water-in-oil emulsions and oil-in-water emulsions), interfacial and in situ polymerization, solvent evaporation from emulsions, desolvation, complex coacervation, polymer and polymer incompatibality, gelation, and pressure extrusion. One of skill in the art will be familiar with each of these classifications.

Schobel, U.S. Pat. No. 4,568,560, discloses encapsulated fragrances and flavors for use in denture cleanser compositions. Schobel discloses encapsulating a solid particulate flavoring agent or fragrance with a film of an acrylic polymer and ethylcellulose. The encapsulation is accomplished utilizing a fluidized bed of the flavoring agent or fragrance.

Yang, U.S. Pat. No. 4,740,376, discloses encapsulating an active ingredient in a solvent free encapsulation composition which includes a blend of a high molecular weight polyvinyl acetate and a hydrophilic plasticizer. The active ingredient is protected from deterioration due to moisture and is provided with controlled release for use in a product to be ingested by a mammal.

Cherukuri et al., U.S. Pat. No. 4,981,698, discloses a delivery system for sweeteners that comprises a first high intensity sweetener encapsulated in a first core coating, and a second outer hydrophilic coating containing up to the solubility limit of the second coating of a second sweetener. The delivery system offers enhanced up front sweetness intensity in combination with prolonged sweetness duration, and improved protection and stability of the sweetener.

Cherukuri et al., U.S. Pat. No. 5,004,595, discloses a free-flowing particulate delivery system for providing enhanced flavor and sweetness to comestible products. The delivery system includes an encapsulating matrix that protects flavor in a core.

Cherukuri et al., U.S. Pat. No. 5,266,335, discloses microencapsulated flavoring agents and methods for preparing the same. The microencapsule comprises a flavoring agent and a resin in the core, and a coating layer over the core. The core is encapsulated by emulsion of a flavoring agent and a resin with a coating layer prepared by complex coacervation of a mixture of two or more colloidal materials.

Kehoe, U.S. Pat. No. 4,975,270, discloses elastomer encased active ingredients. The active ingredients are physically encased in non-porous, chewable particles of elastomer. The particles are then incorporated into articles of commerce.

As is seen above, historically, the most convenient and commonly employed route of drug delivery has been by oral ingestion. The original controlled release of pharmaceuticals was through coated pills which dates back over 1000 years. Coating technology advanced in the mid- to late 1800s with the discovery of gelatin and sugar coatings. A major development in coating technology was the concept of coating drug-containing beads with combinations of fats and waxes. Since the mid-1900s, hundreds of publications and nearly a thousand patents have appeared on various oral delivery approaches encompassing delayed, prolonged, suspended and most recently, controlled release of active ingredients.

In the mid- to late 1960s, the term controlled drug delivery came into being to describe new concepts of dosage form design. These concepts usually involved controlling drug dissolution but also had additional objectives. The primary objectives of a controlled-release system have been to enhance safety and extend duration of action. Today, controlled-release systems are designed in order to produce more reliable absorption and to improve bioavailability and efficiency of delivery.

The overwhelming majority of controlled release systems rely on dissolution, diffusion, or a combination of dissolution and diffusion to generate slow release of a drug. Ueda et al., U.S. Pat. No. 4,874,549, disclose a time-controlled system in which a drug is diffused into a patient after the explosion of a membrane at a given period of time after ingestion. The system is comprised of a preparation in the form of a bead or granule which makes up a core, a drug, a swelling agent and an outer membrane made up of a water-insoluble coating material.

Chen, U.S. Pat. No. 5,508,040, discloses a multiparticulate pulsatile drug delivery system. The system is comprised of a large number of pellets containing a drug and a water soluble osmotic agent. The pellets are an agglomerate of sugar seeds with the drugs spray-coated thereon.

Philippon et al., U.S. Pat. No. 5,229,135, disclose a sustained release diltiazem formulation. The formulation is ingested orally and, like Chen above, the core is a central sugar sphere with a plurality of coatings in which the drug is adhered to the sphere.

Chen, U.S. Pat. No. 5,567,441, also discloses a diltiazem controlled release formulation to be ingested orally. And like Chen and Philippon above, the core is a non-pareil or sugar bead on which the drug is applied via a coating.

However, there are a number of perceived disadvantages with regards to controlled release of drugs into a system. The disadvantages include a longer time to achieve therapeutic blood concentrations, possible increased variation in bioavailability after oral administration, enhanced first-pass effect, dose dumping, sustained concentration in overdose cases, lack of dosage flexibility and, often, greater expense. It should be pointed out that there are a number of constraints on the design of oral controlled drug delivery systems: dose size, drug molecular size, charge and pKa, aqueous solubility, partition coefficient, stability, absorption, metabolism, half-life, margin of safety, toxicity, and clinical response.

In addition, there are a number of disadvantages when using the traditional encapsulation processes to encapsulate active ingredients, including pharmaceuticals and nutriceuticals. The disadvantages include the need for heat and moisture in order to properly form the encapsulated final product. Also, most encapsulation methods are complex and consume large amounts of time in order to obtain the final encapsulated product. Further, current encapsulated ingredients vary in size from nanometers to about 400 microns, and the active ingredients are not uniformly distributed throughout the encapsulated product.

Therefore, there remains a need for an alternate encapsulation method for providing a controlled or extended release product with high levels of active ingredients and in which water is not needed during the encapsulation process, nor is heat an essential feature of the encapsulation process. There also remains a need for an alternate encapsulation method which produces capsules with uniform active ingredient content throughout the product, and that can withstand mechanical pressure both in the processing of the capsule and in the chewing of the product in the mouth so that the active ingredients are released in the stomach of the consumer. Further, there remains a need for a simple encapsulated product that provides good controlled and extended release characteristics for pharmaceuticals.

### BRIEF SUMMARY OF THE INVENTION

Applicant has unexpectedly produced an extended or controlled release encapsulated product, comprising:

a) at least one active ingredient;

b) at least one erodible polymer; and

c) at least one lubricating material; and

d) wherein said product is in the form of a caplet having a diameter from about 1 millimeter to about 7 millimeters and a length from about 1 millimeter to about 7 millimeters.

In a preferred embodiment of the present inventive subject matter, the erodible polymer is a water soluble polymer.

In another preferred embodiment, the erodible polymer is a water insoluble polymer.

A further preferred embodiment is drawn to a pulsating release encapsulated product, comprising:

a) at least one active ingredient;

b) at least two erodible polymers, each of said erodible polymers having a different rate of dissolution; and

c) at least one lubricating material; and

d) wherein said product is in the form of a caplet having a diameter from about 1 millimeter to about 7 millimeters and a length from about 1 millimeter to about 7 millimeters.

A still further preferred embodiment is drawn to a pulsating release product, comprising a capsule having a plurality of caplets, said caplets comprising:

a) at least one active ingredient;

b) at least one erodible polymer;

c) at least one lubricating material; and

d) wherein said caplet has a diameter from about 1 millimeter to about 7 millimeters and a length from about 1 millimeter to about 7 millimeters; and

wherein at least one of said plurality of caplets is prepared from an erodible polymer having a first dissolution rate, and at least another of said plurality of caplets is prepared from another erodible polymer having a second dissolution rate, and said first dissolution rate is not equal to said second dissolution rate.

An advantage of method of the inventive subject matter is that no heat nor moisture is required for forming the encapsulated product. High levels of active ingredients are obtainable in the products of the inventive subject matter, even though heat or moisture is not required for forming the encapsulated product. In addition, the encapsulated product of the present inventive subject matter has a uniform active ingredient content and may be strong enough to withstand mechanical pressure both in the processing of the product, and in the chewing of the product in the mouth so that the active ingredients are released in the stomach.

### DETAILED DESCRIPTION OF THE INVENTION

The encapsulated product of the present invention is a caplet containing a surprisingly high amount of an active ingredient and providing excellent controlled or extended release properties. Applicants have unexpectedly determined that active ingredients can be compressed with high load into a small encapsulated product.

The controlled release products of the present inventive subject matter are designed to produce a sustained concentration of pharmaceutical in the blood. The advantages of the controlled or extended release products of the present inventive subject matter include reduced toxicity and sustained efficacy of the active ingredients; decreased frequency of dosing, resulting in improved patient compliance, reduced patient care; and possibly reduced amount of drug used.

In a preferred embodiment of the present invention, the controlled or extended release encapsulated product of the present inventive subject matter is a caplet shaped like a capsule and having a diameter from about 1 millimeter to about 7 millimeters and a length from about 1 millimeter to about 7 millimeters. Preferably, the diameter of the encapsulated product is about 3 millimeters and the length is about 3 millimeters. The caplets may be coated with a thin surface film to protect the product from moisture or water absorption, from flavor release in the final product system, and from heat and rupture during processing and chewing.

As used herein, the expression “mammal” includes without limitation any mammalian subject, such as mice, rats, guinea pigs, cats, dogs, human beings, cows, horses, sheep or other livestock.

As used herein, the term “active ingredient” includes without limitation: flavors, sweeteners, herbal ingredients, pharmaceuticals, vitamins, minerals, and mixtures thereof.

As used herein, “controlled release” or “extended release” relates to the release rates of the pharmaceutical from the encapsulated product into the mammal. The terms refer to the release of the drug over a period of time, for example from one hour to twenty-four hours.

Currently, controlled release formulations are generally created through different technological approaches which include:

Drug loading on spherical pellets, can be accomplished through either solvent based or aqueous coatings.

Monolithic (tablets) dosage forms based on hydrophyllic swelling polymers in which the drug is dispersed and then released through hydrated swollen matrix.

Erodible matrixes, either multi-particulate or monolithic, that release the included active substance, generally poorly water soluble, by controlled erosion of the system.

Osmotic systems in monolithic, tablets, form that release the drug, soluble in digestive fluids, through a calibrated hole in the osmotic membrane surrounding the tablet.

In a preferred embodiment, the active ingredient to be released into a mammal is incorporated into an erodible polymer matrix. A general method for preparing a controlled-release encapsulated product encompasses the following steps. First, the active ingredient is mixed with a suitable erodible polymer. The active ingredient may be present in amounts from 0.001 to 70.0% by weight of the final encapsulated product. The erodible polymer may be present from 10.0 to 70.0% by weight of the final encapsulated product.

The present inventive subject matter contemplates that the erodible polymer may be either water soluble or water insoluble. Water soluble polymers useful in the present inventive subject matter include, without limitation, sodium carboxymethyl cellulose, hydroxyethyl cellulose, hydroxypropyl cellulose, propylene glycol alginate, sodium alginate, carboxymethyl cellulose and mixtures thereof.

Likewise, water insoluble polymers that are useful in the present inventive subject matter include, without limitation, cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, and mixtures thereof.

The active ingredient/polymer mixture is then granulated using a suitable binder. The binder is generally present in amounts of 1.0 to 10.0% by weight of the final encapsulated product. Binders suitable for use in the present inventive subject matter include, without limitation, plasdone K-29/32, povidone K30, carboxymethylcellulose sodium, ethylcellulose, methylcellulose, alginic acid and mixtures thereof.

After the active ingredient/polymer mixture is granulated, the mixture is passed through a mesh, preferably a mesh no. 10, and allowed to air dry. After air-drying, the mixture is passed through another mesh, preferably a no. 20 mesh.

After passing through the second mesh, the granulated mixture is lubricated with a lubricant and compressed into capsules with the sizes listed above. The lubricant or lubricating material forms a film around the granules and helps the material flow, compress and eject from the tableting machine. The lubricant or lubricating material may be present in levels up to 5% by weight of the final composition. Examples of usable lubricating materials include, without limitation, fats, emulsifiers, waxes, magnesium stearate, calcium stearate, talc, starches, silicon dioxide, and mixtures thereof. Among the fats, or fatty materials, useful herein include, without limitation, water-insoluble, inert hydrocarbon fats or oils, or their derivatives and mixtures thereof. Such fats or fatty materials include, for example and without limitation, cocoa butter, hydrogenated vegetable tallow, hydrogenated vegetable oils, and derivative mixtures thereof.

Among the emulsifiers useful herein include, without limitation, alkyl aryl sulfonates, alkyl sulfates, sulfonated amides and amines, sulfated and sulfonated esters and ethers, alkyl sulfonates, polyethoxylated esters, mono- and diglycerides, diactyl tartaric esters of monoglyderides, polyglycerol esters, sorbitan esters and ethoxylates, lactylated esters, propylene glycol esters, sucrose esters and mixtures thereof. Among the waxes useful herein include, without limitation, amorphous waxes, anionic emulsifying waxes, bleached waxes, caranda waxes, cetyl esters, cationic emulsifying waxes, microcrystalline waxes, paraffins, refined waxes and mixtures thereof.

The use of particular fats, emulsifiers or waxes may allow the encapsulated product of the present inventive subject matter to aid in providing controlled or sustained release of the active ingredient. The controlled release occurs due to the entrapment of the active material in the particular fat, emulsifier or wax.

It is possible to provide a coating on the encapsulated product. The coating provides protection of the active ingredients from moisture or water absorption. The coating may also allow the release of the active ingredient in the stomach of the individual, and not in the mouth thereof.

In one aspect of the inventive subject matter the encapsulated product is coated with a polymeric coating to form an extended release formulation. In this aspect the extended release formulations are where the mechanism of release is driven, predominantly, by the osmotic pressure.

In the present procedure, the encapsulated products are formulated with osmotic ingredients and coated with semi-permeable film forming polymers to achieve zero-order release. The advantages of this formulation include the combination of the mechanism of the control of the release based on the osmotic pressure (finely tuned and independent from the motility, pH, composition of the digestive fluids and food) with the concept of multiple units (improved inter and intra-subjects variability of absorption).

Active ingredients can be any pharmacologically active substance to be administered by the oral route and included in a solid dosage form.

Osmotic ingredients can be any active principle ingredient with a solubility in aqueous media, in the range of pH from 1 to 7 and in an amount of not less than 0.1%.

Any inorganic salt may be used which is highly dissociated in aqueous media in the range of pH from 1 to 7 and suitable to be included in pharmaceutical preparations for oral administration.

Semi-permeable film forming polymers can be high molecular weight derivatives of cellulose which are insoluble in water as ethylcellulose with a degree of ethylation between 43% and 50%, cellulose acetate with 30%-45% of acetyl value, polyvinylacetate, ammonium methacrylate co-polymers.

Suitable plasticizers can be added in the range of 5% to 35%. The film thickness may vary from 20 μm to 100 μm to achieve the desired extended release profile. The size of encapsulated products may vary between 2 mm to 3 mm of diameter and height. Depending on the composition of the core and on the type and thickness of the film different zero order kinetics can be achieved.

This encapsulated product formulation can be applied to any controlled release applications including OTC & Rx Pharmaceuticals, and Nutritional applications.

In another aspect of the inventive subject matter, the encapsulated product is coated with a polymeric coating to form a delayed release formulation. In this aspect, the delayed release, monolithic, oral dosage form is based on osmotic pressure and then a crown coating in the coating polymer formed in situ when the dose is ingested.

Oral dosage forms have heretofore been generally based on osmotic pressure by preparing a core, usually in the form of a tablet, containing the active substance dispersed in a combination of ingredients able to generate an osmotic pressure, when contained by an osmotic membrane. The release from such systems is obtained with a calibrated hole in the membrane which regulates, together with the level of osmotic pressure generated by the intrinsic characteristics of the tablet core, the rate of release.

In the present invention, tablets with swelling polymers, osmotic ingredients and active substance, with a cone protuberance on one side are then coated with semi-permeable film-forming polymer. The thickness of the film is lower on the cone. By swelling, the film brakes on the cone because the lower thickness and the content of the vesicle (the coated tablet) is released. For given dimensions and composition of the tablet, the time to break is regulated by the thickness of the film and the height of the cone. This system allows to match the result without the need of holing the osmotic film with a laser beam, risk of irradiated polymers with free radicals, or by using a micro drill, a costly process.

Active ingredients can be any pharmacologically active substance to be administered by the oral route and included in a solid dosage form.

Osmotic ingredients can be any active principle ingredient with a solubility in aqueous media, in the range of pH from 1 to 7 and in amounts not less than 0.1%.

Any inorganic salt may be used which is highly dissociated in aqueous media in the range of pH from 1 to 7 and suitable to be included in pharmaceutical preparations for oral administration.

Swelling polymers can be polycarbophyls and hydroxypropylmethylcellulose of different viscosity such as from 4,000 to 100,000 cps.

The diameter of the tablets can vary from 5 mm to 10 mm.

The thickness can vary from 3 mm to 5 mm.

The height of the cone can vary from 0.5 mm to 1.0 mm.

The shape of the protuberance is conical with the diameter of the base that can vary from 0.8 to 1.3 mm.

Semi-permeable film forming polymers can be high molecular weight derivatives of cellulose which are insoluble in water as ethylcellulose with a degree to ethylation between 43% and 50%, cellulose acetate with 30%-45% of acetyl value, polyvinylacetate, ammonium methacrylate co-polymers.

Suitable plasticizers can be added in the range of 5% to 35%. The film thickness may vary from 20 μm to 150 μm.

This formulation can be applied to any modified release applications including OTC & Rx Pharmaceuticals, and Nutritional applications.

In a preferred embodiment of the present inventive subject matter, the controlled release of the encapsulated product provide “pulses” or “pulsating release” of the active ingredients. By “pulses” or “pulsating release”, Applicants mean that the active ingredient is released at different time intervals while in the body of the mammal. By “pulsating,” Applicants also mean that the release of the active ingredients may be continuous, discontinuous, extended or sustained. Preferably, the “pulsating” aspect of the release of the active ingredients means the discontinuous release of the actives.

Many active ingredients need frequent administration of burst doses in order to achieve optimal effect of the active ingredient. The most common way of achieving pulsating release of active ingredients has been to coat the drug with slowly dissolving polymeric membranes or with protective polymers that dissolve selectively at pH's corresponding to specific regions of the gastrointestinal tract. Once the polymeric membrane has dissolved, all of the drug inside the membrane is immediately available for dissolution and absorption. Thus, the drug release can be controlled by adjusting the thickness and dissolution rate of the polymeric membrane surrounding the drug. If only a few different thicknesses of membrane are used, the drug will be released at different, predetermined times, or “pulses.” The present inventive subject contemplates coating the encapsulated products with such polymeric membranes, as is discussed above.

In addition, however, the present inventive subject matter provides pulsating delivery of the active ingredient by taking advantage of the different characteristics of the different the polymer erodible polymers used in the encapsulated products. In a preferred embodiment of the present inventive subject matter, the encapsulated product is prepared with at least two erodible polymers, each having a different rate of dissolution in the body of the mammal in which the encapsulated product is introduced.

For example, the encapsulated product may be made with two or more erodible polymers, at least one that erodes quickly in the body to provide immediate dissolution of the drug and at least another that does not erode as quickly, thus delaying release of the active ingredient until a desired time.

As is stated above, an important aspect of this embodiment of the present inventive subject matter is the incorporation of at least two erodible polymers having different rates of dissolution. The present inventive subject matter contemplates the use of both water soluble polymers and water insoluble polymers for this preferred embodiment. Examples of water soluble and water insoluble polymers are listed above. Depending on the desired characteristics encapsulated product and release profile of the active ingredient, the erodible polymers used to achieve the pulsating release of the active ingredient may be water soluble, water insoluble, or a mixture thereof. One of ordinary skill in the art will be able to easily determine which polymers are suitable to achieve the desired pulsating release of the active ingredients based on the dissolution rates of the various erodible polymers.

In a further embodiment of the present inventive subject matter, the pulsating effect is achieved by incorporating into a standard capsule encapsulated products prepared from different erodible polymers. In this embodiment, the active ingredient is incorporated into multiple encapsulated products using two or more different erodible polymers, with each encapsulated product being prepared with a different erodible polymer. Then, the different encapsulated products are included in a standard capsule which is taken by the mammal. The encapsulated products will erode at different rates based upon the erodible polymers with which the products were made, providing a pulsating release profile of the active ingredients. Again one of ordinary skill in the art will be able to easily determine which polymers are suitable to achieve the desired pulsating release of the active ingredients based on the dissolution rates of the various erodible polymers.

While the above delivery of controlled release encapsulated products is by oral ingestion of the encapsulated products, the present inventive subject matter also contemplates site-specific delivery of the active ingredients by different modes of introduction of the active ingredient into the body. The different modes include, for example, introduction of the encapsulated products rectally, which will allow introduction of the products directly into the large bowel of the mammal. In this way, the encapsulated products will act much like a suppository, providing controlled release of the active ingredients while at the same time by-passing the oral route of delivery.

Another example of a non-oral controlled delivery of active ingredients by the present inventive subject matter is to have the inventive encapsulated products implanted into the body. In this embodiment, the encapsulated products, produced and preserved sterile until use, would be implanted directly into the region of the body where the need for the active ingredient is the greatest. Then, as the bodily fluids erode the erodible polymers of the encapsulated products, the active ingredients would be released directly to the site which needs the active ingredients the most.

A further example of site specific delivery of active ingredients according to the present inventive subject matter is to apply the inventive encapsulated products directly to a cut or abrasion on the skin of the mammal being treated. The size of the inventive encapsulated products allows for direct application to the wound in order to directly deliver the needed active ingredient. Preferably, the inventive encapsulated products will be held in place with a bandage, thus keeping optimal contact between the encapsulated products and the wound.

In the above non-oral delivery embodiments, the controlled release encapsulated products may have the same characteristics as described above for oral delivery of active ingredients. That is, the controlled release may be zero order, or may provide a pulsating effect, as is defined above. Further, the pulsating effect may be the result of two or more different erodible polymers being incorporated into the same inventive encapsulated product, or the pulsating effect may be due to the presence of multiple encapsulated products having been made from different erodible polymers having different rates of dissolution.

The above non-oral delivery routes of active ingredients are meant as non-limiting examples only, and it should be recognized that other non-oral delivery routes are also within the contemplation of the present inventive subject matter.

The amount of active material present in the inventive compositions will vary depending on the particular active used, but generally will be present in an amount of about 0.001% to 70% by weight of the composition. Preferably, the active ingredients used in the inventive compositions are prophylactic or therapeutic active ingredients. Prophylactic or therapeutic active materials which can be used in the present invention are varied. A non-limiting list of such materials includes the following: antibiotics, antitussives, antihistamines, decongestants, alkaloids, mineral supplements, laxatives, antacids, ion exchange resins, anti-cholesterolemics, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs and mixtures thereof.

Preferred therapeutic active materials contemplated for use in the present inventive subject matter are analgesics. Examples of analgesics useful in the present inventive subject matter, and which are the preferred therapeutic active ingredients, include, without limitation, aspirin, acetaminophen, ibuprophen and mixtures thereof.

Another preferred active material can be selected from the class of prophylactic, abortive or analgesic drugs used to treat migraines. Migraines are defined as headaches that last 4 to 72 hours wherein the patient experiences moderate to severe cranial throbbing. Migraines are also associated with nausea, vomiting, or sensitivity to light, sound or smell.

For prophylactic treatment of migraines, β-blockers, calcium channel blockers, tricyclic antidepressants, or anticonvulsants can be used. Examples of drugs indicated for prophylactic treatment include amitriptyline, methysergide, popranolol, valproate, and verapamil.

For abortive treatment of migraines serotonin receptor activators such as eletriptan, ergotamine, naratriptan, rizatriptan benzoate, sumatriptan succinate, and zolmitriptan can be used. Ergot alkaloid derivatives such as ergoamine tartrate and dihydroergotamine are also effective. Dopamine antagonist anti-emetics such as dimenhydrinate, metoclopramide and prochlorperazine, while indicated for the treatment of nausea, can also be used even if nausea is not prominent.

For analgesic treatment acetaminophen, aspirin, non-asteroidal anti-inflammatory drugs (“NSAID”) and opioids can be used in the present invention.

In general, any class of drug indicated for migraine treatment may be used in the present invention. For example, sumatriptan succinate may be incorporated into the encapsulated products of the present invention to effectively deliver sumatriptan succinate to a patient in need thereof. In particular, sumatriptan succinate can be formulated with the present invention in doses ranging from 25, 50, to 100 mg daily. All the examples are non-limiting and it will be understood that other migraine therapeutics may be used with the present inventive subject matter.

Yet another preferred active material used in the composition of the present inventive matter is a psychotropic. Psychotropics are used to treat depression, schizophrenia, anxiety disorders, attention deficit order, obsessive compulsive disorder, senile dementia and certain sleep disorders.

The classes of drugs used in treating depression include selective serotonin reuptake inhibitors (“SSRI's”), heterocyclic antidepressants, monoamine oxidase inhibitors (“MAOI's”), serotonergic-noradrenergics, 5-HT₂ antagonists and catecholaminergics. Examples of SSRI'S include fluoxetine HCl, sertraline HCl, paroxetine HCl, and fluvoxamine. Examples of heterocyclic antidepressants include amitriptyline, nortriptyline, imipramine, desipramine, doxepin, trimipramine, clomipramine, protriptyline, amoxapine, and maprotiline. Examples of MAOI's include phenelzine and tranylcypromine. An example of a serotonergic-noradrenergics includes venlafaxine HCl. Examples of 5-HT₂ antagonists include trazadone, nefazodone, and mirtazapine. An example of a catecholaminergics includes bupropion. All examples are non-limiting and it will be understood that psychotropics of the disclosed classes may be used with the present inventive subject matter.

In general, any class of psychotropic drug indicated for treating depression may be used in the present invention. For example, fluoxetine HCl may be incorporated into the encapsulated products of the present invention to effectively deliver fluoxetine HCl to a patient in need thereof. In particular, fluoxetine HCl can be formulated with the present invention in doses ranging from about 10 to 60 mg daily. One of ordinary skill in the art will be able to determine the proper dosage for the remaining disclosed drugs.

For the treatment of anxiety, benzodiazepines may be used with the present inventive subject matter. Specific examples include alprazolam, chlordiazepoxide, clonazepam, clorazepate, diazepam, lorazepam, and oxazepam. However, any class of psychotropic drug indicated for anxiety treatment may be used in the present invention.

In particular, alprazolam may be incorporated into the encapsulated products of the present invention to effectively deliver alprazolam to a patient in need thereof. In particular, alprazolam can be formulated with the present invention in doses ranging from about 0.25 to 0.50 mg to be taken three times daily. One of ordinary skill in the art will be able to determine the proper dosage for the remaining disclosed drugs.

For the treatment of insomnia, drugs belonging to the categories of benzodiazepines, imidazopyridines, antidepressants and non-prescription hypnotics may be used with the present inventive subject matter. Examples of benzodiazepines useful for the treatment of insomnia include midazolam, triazolam, oxazepam, temazepam, lorazepam, estazolam, nitrazepam, diazepam, quazepam, flurazepam, zopiclone and clorazepate. An example of an imidazopyridine includes zolpidem and zolpidem tartarate. Examples of antidepressants include amityiptyline and doxepin.

In particular, zolpidem may be incorporated into the encapsulated products of the present invention to effectively deliver zolpidem to a patient in need thereof. In particular, zolpidem can be formulated with the present invention in doses ranging from about 5.0 to 30.0 mg daily, the preferred range being from about 5.0 to 10.0 mg daily. One of ordinary skill in the art will be able to determine the proper dosage for the remaining disclosed drugs. Moreover, all the examples are non-limiting and it will be understood that other psychotrpoics may be used with the present inventive subject matter.

Still yet another preferred active material used in the composition of the present inventive matter is a gastrointestinal therapeutic. Gastrointestinal therapeutics are used to treat gastritis, nausea and vomiting, gastroesophegal reflux disease, colitis, Crohn's disease and diarrhea. Classes of drugs include proton pump inhibitors, histamine H₂ receptor antagonists, terpene analogs, and NSAID'S.

For the treatment of gastritis, drugs such as omeprazole, lansoprazole, ranitidine HCl, famotidine, nizatidine, teprenone, cimetidine, rabeprazole sodium, and sulpiride can be used in the compositions of the present inventive subject matter.

For the treatment of nausea and vomiting, drugs such as ondansetron HCl, granisetron HCl, dolasetron mesylate, and tropisetron may be used.

In particular, omeprazole may be incorporated into the encapsulated products of the present invention to effectively deliver omeprazole to a patient in need thereof. In particular, omeprazole can be formulated with the present invention in doses ranging from about 10.0 to 60.0 mg daily, the preferred range being from about 15.0 to 25.0 mg daily. One of ordinary skill in the art will be able to determine the proper dosage for the remaining disclosed drugs. Moreover, all the examples are non-limiting and it will be understood that other gastrointestinal therapeutics may be used with the present inventive subject matter.

Another preferred active material used in the compositions of the present invention include cardiovascular therapeutics. Cardiovascular therapeutics treat hypertension, angina, myocardial infarction, congestive heart failure, acute coronary syndrome, edema, ventricular tachycardia, hyperaldosteronism, ventricular arrhythmia, cardiac insufficiency, atrial fibrillation, arterial occlusion, cardiac decompensation, and microcirculation activation.

A related class of cardiovascular therapeutics are cholesterol reducers such as 3-hydroxy-3-methylglutaryl coenzymeA (“HMG-CoA”) reductase inhibitors. HMG-CoA inhibitors work by blocking an enzyme used to make cholesterol. Blocking cholesterol thereby treats hypercholesterolemia which is a significant cause of cardiovascular disease.

For the treatment of hypercholesterolemia, drugs such as simvastin, atorvastatin calcium, pravastatin sodium, pravastatin, lovastatin, fluvastatin sodium, cerivastatin sodium can be used in the compositions of the present inventive subject matter.

For the treatment of hypertension, drugs such as nifedipine, amlodipine besylate, losartan potassium, lisinopril, felodipine, benazepril HCl, ramipril, irbesartan, verapamil HCl, bisoprolol fumarate and hydrochlorothiazide, amlodipine and benazepril HCl, clonidine, candesartan, cilexetil, diltiazem, nicardipine, imidapril, trandolapril, eprosartan mesylate, nilvadipine, verapamil HCl, temocapril, prazosin HCl, isradipine, cilazapril, celiprolol, bisoprolol, betazolol HCl, ramipril, nisoldipine, lisinopril, trandolapril, and nisoldipine can be used in the compositions of the present inventive subject matter.

For the treatment of congestive heart failure, drugs such as dioxin, carvedilol, spironolactone, trandolapril, and bisoprolol can be used in the compositions of the present inventive subject matter.

In particular, simvastin may be incorporated into the encapsulated products of the present invention to effectively deliver simvastin to a patient in need thereof. In particular, simvastin can be formulated with the present invention in doses ranging from about 5.0 to 80 mg daily. One of ordinary skill in the art will be able to determine the proper dosage for the remaining disclosed drugs. Moreover, all the examples are non-limiting and it will be understood that drugs from the disclosed classes may also be used with the present inventive subject matter.

Still another preferred active material used in the composition of the present invention is a therapeutic useful for treating allergic rhinitis. The classes of compounds useful for treating allergic rhinitis include alkylamines, ethanolamines, ethylenediamines, piperazines, phenothiazine, piperdines, and nonsedating compounds.

Among the non-sedating compounds that can be used in the present invention are loratadine, fexofenadine HCl, certirizine HCl, and astemizole. Other drugs which can also be used are fluticasone propionate, mometasone furoate, epinastine, beclomethasone dipropionate, triamcinolone acetonide, budesonide, and azelastine.

In particular, loratadine may be incorporated into the encapsulated products of the present invention to effectively deliver loratadine to a patient in need thereof. In particular, loratadine can be formulated with the present invention in doses ranging from about 5.0 to 15 mg daily, with 15 mg daily being the preferred dosage. One of ordinary skill in the art will be able to determine the proper dosage for the remaining disclosed drugs. Moreover, all the examples are non-limiting and it will be understood that other allergic rhinitis therapeutics may be used with the present inventive subject matter.

Still yet another preferred active material used in the composition of the present invention is a therapeutic useful for treating osteoarthritis or rheumatoid arthritis. Rheumatoid arthritis is defined as non-specific, symmetrical inflammation of the peripheral joints, potentially resulting in progressive destruction of articular and periarticular structures. Osteoarthritis is characterized by loss of articular cartilage and hypertrophy of bone. Although osteoarthritis is a degenerative bone disease, symptoms associated with rheumatoid arthritis such as inflammation of the joints occur in a patient diagnosed with osteoarthritis. Accordingly, therapeutics treating rheumatoid arthritis can also be administered to an osteoarthritic patient.

Classes of drugs indicated for osteoarthritis and rheumatoid arthritis include cycloxygenase-2 inhibitors, NSAID'S, biologic response modifiers, pyrimidine synthesis inhibitors and hyaluronic acid. Specific examples of osteoarthritis and rheumatoid arthritis therapeutics include celecoxib, diclofenac sodium, rofecoxib, nabumetone, diclofenac sodium and misoprostol, oxaprozin, meloxicam, piroxicam, etodolac, naproxen, hylan G-F 20, leflunomide, tenoxicam, and naproxen sodium.

In particular, celecoxib may be incorporated into the encapsulated products of the present invention to effectively deliver celecoxib to a patient in need thereof. In particular, celecoxib can be formulated with the present invention in doses ranging from about 150 to 250 mg daily, with 200 mg daily being the preferred dosage. One of ordinary skill in the art will be able to determine the proper dosage for the remaining disclosed drugs. Moreover, all the examples are non-limiting and it will be understood that other osteoarthritis and rheumatoid arthritis therapeutics from the disclosed classes may also be used with the present inventive subject matter.

Another preferred active material used in the composition of the present invention is a therapeutic useful for treating benign prostatic hypertrophy. Benign prostatic hypertrophy is defined as an adenomatous hyperplasia of the periurethral part of the prostrate gland.

Classes of drug useful for the treatment of benign prostatic hypertrophy include alpha blockers, alpha-1 selective adrenoceptor blocking agents and 5-reductase inhibitors. Specific examples of benign prostatic hypertrophy therapeutics include doxazosin mesylate, terazosin HCl, tamsulosin, finasteride, tamsulosin HCl, ethinyl estradiol and levonorgestrel.

In particular, doxazosin mesylate may be incorporated into encapsulated products of the present invention to effectively deliver doxazosin mesylate to a patient in need thereof. In particular, doxazosin mesylate can be formulated with the present invention in doses ranging from about 1 to 16 mg daily. One of ordinary skill in the art will be able to determine the proper dosage for the remaining disclosed drugs. Moreover, all the examples are non-limiting and it will be understood that other benign prostatic hypertrophy therapeutics from the disclosed classes may also be used with the present inventive subject matter.

Yet another preferred active material used in the composition of the present invention is a drug indicated for the treatment of fungal infections. Classes of drugs indicated for the treatment of fungal infections include synthetic triazole, ergosterol inhibitor, and polyene antifungal. Specific examples of drugs indicated for the treatment of fungal infections are itraconazole, ketoconazole, and amphotericin B.

In particular, itraconazole may be incorporated into the encapsulated products of the present invention to effectively deliver itraconazole to a patient in need thereof. In particular, itraconazole can be formulated with the present invention in doses ranging from about 1.0 to 400 mg daily. One of ordinary skill in the art will be able to determine the proper dosage for the remaining disclosed drugs. Moreover, all the examples are non-limiting and it will be understood that other anti-fungals from the disclosed classes may also be used with the present inventive subject matter.

Still yet another preferred active material used in the composition of the present invention is a anti-convulsant. Anti-convulsants are drugs that prevent or relieve convulsions wherein the convulsions are due to epilepsy, seizure disorders, partial seizure disorders or Huntington's disease. Classes of drugs useful for treating these conditions include gamma-aminobutyric analogs, phenyltriazine, antiepileptic agents, benzodiazepines, polysynaptic response inhibitors, sulfamate-substituted monosaccharides, gamma-amino butyric acid uptake inhibitors and benzamides. Specific examples include carbamazepine, topiramate, and tigabine HCl.

In particular, carbamazepine may be incorporated into the encapsulated products of the present invention to effectively deliver carbamazepine to a patient in need thereof. In particular, carbamazepine can be formulated with the present invention in doses ranging from about 100 to 1600 mg daily. One of ordinary skill in the art will be able to determine the proper dosage for the remaining disclosed drugs. Moreover, all the examples are non-limiting and it will be understood that other anti-convulsants from the disclosed classes may also be used with the present inventive subject matter.

Another preferred active material used in the composition of the present invention is an anti-herpetic. Anti-herpetics are used to treat infections from the varicella-zoster virus. Classes of drugs useful for treating herpes include synthetic purine nucleoside analogs, nucleoside analogs, and antiviral agents. Specific examples include acyclovir, valacyclovir HCL and famcyclovir.

In particular, acyclovir may be incorporated into the encapsulated products of the present invention to effectively deliver acyclovir to a patient in need thereof. In particular, acyclovir can be formulated with the present invention in doses ranging from about 200 to 800 mg daily. One of ordinary skill in the art will be able to determine the proper dosage for the remaining disclosed drugs. Moreover, all the examples are non-limiting and it will be understood that other anti-herpetics from the disclosed classes may also be used with the present inventive subject matter.

Yet another active material used in the compositions of the present invention are anti-diarrheal therapeutics. Anti-diarrheal therapeutics treat the condition of diarrhea whether it is symptomatic of the disorder itself wherein diarrhea is a condition that occurs when a mammal has a low amount of stool in a bowel movement. Diarrhea results mainly from excess fecal water in the bowel of the mammal. Specific examples of anti-diarrheal therapeutics include loperamide HCl, diphenoxylate, codeine phosphate, camphorated opium tincture.

In a further embodiment of the present invention, he encapsulated product includes the incorporation of flavors. The flavoring agents which may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture.

Other useful flavorings include aldehydes and esters such as cinnamyl acetate, cinnamaldehyde, citral diethylacetal, dihydrocarvyl acetate, eugenyl formate, p-methylamisol, and so forth may be used.

If the flavor to be added is liquid, then the liquid flavor is first absorbed onto a solid absorbent. Examples of absorbents on which the liquid may be absorbed include, without limitation, silica gel particles, starches, carbohydrates such as sugars and polyhydroxyalcohols, celluloses, calcium salts such as calcium phosphate, calcium carbonate, and calcium sulfonate, and other absorbing agents in free-flowing powder form. The amount of liquid flavor added depends on the final concentration desired. Generally, though, the liquid flavor will be present in quantities from about 0.1% to 70% by weight of the resultant flavor/absorbent mixture.

The flavor/absorbent mixture is then mixed with a the erodible polymer above.

Furthermore, other additives such as colors, may also be added to this mixture to form the final mixture. The final mixture is then formed into the encapsulated product of the present invention by using a tableting machine. The stations of the tableting machine are set to the desired caplet size, which is from about 1 millimeter to about 7 millimeters diameter and length for the encapsulated.

The use of flavor with the active ingredient in the encapsulated product allows for flexibility in adding flavor to food items, confectionery products or chewing gum products. For example, delivery of two or more flavors to a single food item is possible by using encapsulated products containing different flavors in the food item. The delivery of two or more flavors is also possible in confectionery products and chewing gum products.

Advantages of preparing the inventive encapsulated product in this manner are that no heat and no moisture are needed in this process. Additionally and surprisingly, high concentrations of active ingredients may be incorporated into the final encapsulated product. Furthermore, the encapsulated product of the present inventive subject matter is small enough that when the confectionery or chewing gum product is chewed, the encapsulated product can pass with the saliva and not be disformed by the teeth of the individual chewing.

Examples of vitamins that are available as active ingredients include, without limitation, vitamin A (retinol), vitamin D (cholecalciferol), vitamin E group (α-tocopherol and other tocopherols), vitamin K group (phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof. The amount of vitamin or vitamins present in the final encapsulated product of the present inventive subject matter is dependent on the particular vitamin and is generally the United States' Department of Agriculture Recommended Daily Allowances (USRDA) for that vitamin. For example, if vitamin C is the active ingredient and the encapsulated product is being used in a confectionery or chewing gum targeting adults, the amount of vitamin C in the encapsulated product would be 60 milligrams, which is the USRDA of vitamin C for adults.

Examples of minerals that are available as active ingredients include, without limitation, calcium, magnesium, phosphorus, iron, zinc, iodine, selenium, potassium, copper, manganese, molybdenum and mixtures thereof. As is the case with vitamins, the amount of mineral or minerals present in the final encapsulated product of the present inventive subject matter is dependent on the particular mineral and is generally the USRDA for that mineral. For example, if iodine is the active ingredient and the encapsulated product is being used in a confectionery or chewing gum targeting adults, the amount of iodine in the encapsulated product would be 150 micrograms, which is the USRDA of iodine for adults.

Examples of herbals that are available as active ingredients include, without limitation, echinacea, peppermint, licorice, goldenseal, panax pseudoginseng, grapeseed extract, bilberry, kava, ginko biloba, panax quinquefolium, Siberian ginseng, St. John's wort, bromelian, guglupids, hawthorn, garlic, ginger, angelica species, dandelion, goldenseal, and mixtures thereof. Further, examples of spices that are available as active ingredients include, without limitation, mustard, dillweed, cinnamon, garlic, black pepper, onion, sage, oregano, basil, cream of tartar, targon, cayenne pepper, red pepper, and mixtures thereof. This list of herbals and spices is for exemplary purposes and is not meant to be construed as limiting the inventive subject matter thereto.

As is stated above, an advantage of method of the inventive subject matter is that no heat nor moisture is required for forming the encapsulated product. In addition, the encapsulated product of the present inventive subject matter has a uniform active ingredient content and may be strong enough to withstand mechanical pressure both in the processing of the product, and in the chewing of the product in the mouth so that the active ingredients are released in the stomach.

The following examples are illustrative of preferred embodiments of the invention and are not to be construed as limiting the invention thereto. All percentages are given in weight percent, unless otherwise noted and equal a total of 100%.

### EXAMPLES

### Example 1

### Preparation of a 50-mg Capsule of Dimenhydrinate Encapsulated Product

43.48% dimenhydrinate was mixed with 51.31% hydroxypropyl methyl cellulose in a shear mixer. The mixture was then granulated using 3.91% povidone K30 dissolved in isopropyl alcohol. The granulated mixture was passed through a no. 10 mesh and allowed to air dry. When the granulated mixture was dry, it was then passed through a no. 20 mesh. The mixture was next lubricated with 1.30% magnesium stearate. The final mixture was mixed for 3 minutes. The mixture was loaded into a tableting machine.

A series of caplets was produced using 20 KN of force. Each caplet weighed 19 mg and six caplets were filled in a no. 2 capsule to obtain a 50-mg dimenhydrinate capsule.

The 50-mg capsule was then tested in vitro for dissolution characteristics. The capsule was loaded into a no. 2 apparatus (paddle, USP) with 900 ml of water. The capsule was rotated at 50 rpm (revolutions per minute) and the amount dissolved was determined at 30, 60, 120, 180, 240, 360 and 480 minutes.

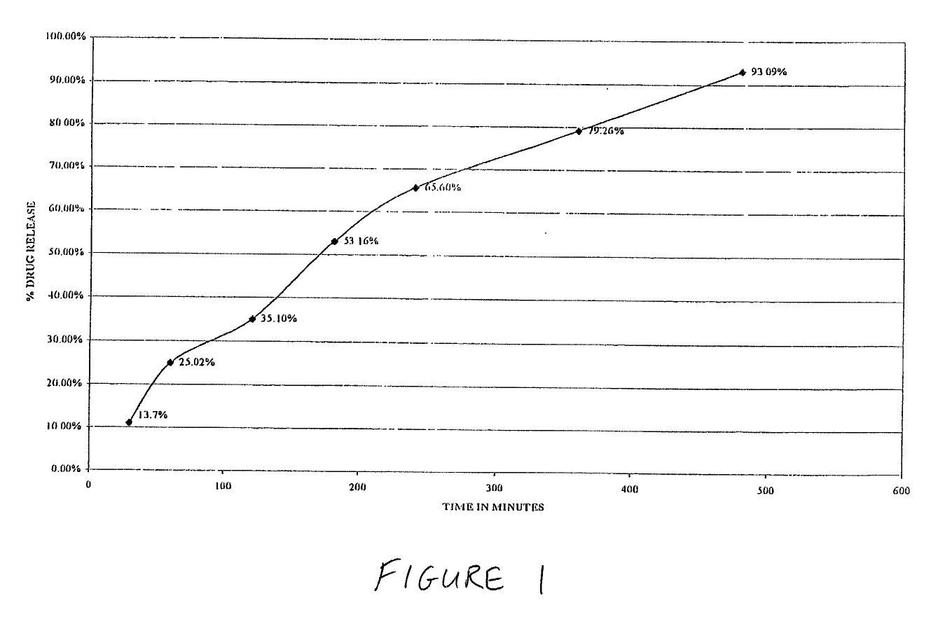

The results of the dissolution test are provided in FIG. 1. As can be seen, the dissolution rate was zero order for the capsule.

### Example 2

### Preparation of a 30-mg Capsule of Nifedipine

34.10% nifedipine was mixed with 10.90% lactose anhydrous, 0.90% microcrystalline cellulose, and 48.90% hydroxypropyl methyl cellulose in a shear mixer. The mixture was then granulated using 4.20% povidone K30 dissolved in isopropyl alcohol. The granulated mixture was passed through a no. 10 mesh and allowed to air dry. When the granulated mixture was dry, it was then passed through a no. 20 mesh. The mixture was next lubricated with 1.00% magnesium stearate. The final mixture was mixed for 3 minutes. The mixture was loaded into a tableting machine.

A series of caplets was produced using 20 KN of force. Each caplet weighed 17.60 mg and five caplets were filled in a no. 2 capsule to obtain a 30-mg nifedipine capsule.

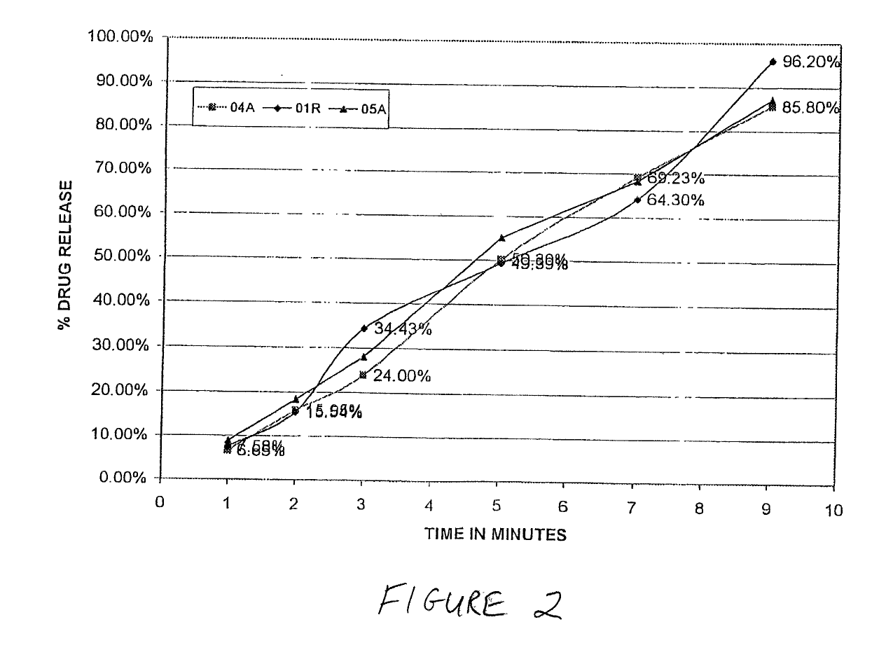

The 30-mg capsule was then tested in vitro for dissolution characteristics. The capsule was loaded into a no. 2 apparatus (paddle, USP) with 900 ml of water (phosphate buffer pH: 6.8). The capsule was rotated at 50 rpm (revolutions per minute) and the amount dissolved was determined at 1, 2, 5, 7 and 9 hours The results of the dissolution test are provided in FIG. 2. As can be seen, the dissolution rate was zero order for the capsule.

### Example 3

### Preparation of a 30-mg Capsule of Nifedipine

34.10% nifedipine was mixed with 20.00% lactose anhydrous, 1.00% microcrystalline cellulose, and 38.90% hydroxypropyl methyl cellulose (19.45% HPMC K15 M and 19.45% HPMC K100 M) in a shear mixer. The mixture was then granulated using 5.00% povidone K30 dissolved in isopropyl alcohol. The granulated mixture was passed through a no. 10 mesh and allowed to air dry. When the granulated mixture was dry, it was then passed through a no. 20 mesh. The mixture was next lubricated with 1.00% magnesium stearate. The final mixture was mixed for 3 minutes. The mixture was loaded into a tableting machine.

A series of caplets was produced using 20 KN of force. Each caplet weighed 17.57 mg and five caplets were filled in a no. 2 capsule to obtain a 30-mg nifedipine capsule.

The 30-mg capsule was then tested in vitro for dissolution characteristics. The capsule was loaded into a no. 2 apparatus (paddle, USP) with 900 ml of water (phosphate buffer pH: 6.8). The capsule was rotated at 50 rpm (revolutions per minute) and the amount dissolved was determined at 1, 2, 5, 7 and 9 hours

The results of the dissolution test are provided in FIG. 2. As can be seen, the dissolution rate was zero order for the capsule.

### Example 4

### Preparation of a 30-mg Capsule of Nifedipine

34.10% nifedipine was mixed with 20.00% lactose anhydrous, 1.00% microcrystalline cellulose, and 38.90% hydroxypropyl methyl cellulose (11.67% HPMC K15 M and 27.23% HPMC K100 M) in a shear mixer. The mixture was then granulated using 5.00% povidone K30 dissolved in isopropyl alcohol. The granulated mixture was passed through a no. 10 mesh and allowed to air dry. When the granulated mixture was dry, it was then passed through a no. 20 mesh. The mixture was next lubricated with 1.00% magnesium stearate. The final mixture was mixed for 3 minutes. The mixture was loaded into a tableting machine.

A series of caplets was produced using 20 KN of force. Each caplet weighed 17.57 mg and five caplets were filled in a no. 2 capsule to obtain a 30-mg nifedipine capsule.

The 30-mg capsule was then tested in vitro for dissolution characteristics. The capsule was loaded into a no. 2 apparatus (paddle, USP) with 900 ml of water (phosphate buffer pH: 6.8). The capsule was rotated at 50 rpm (revolutions per minute) and the amount dissolved was determined at 1, 2, 5, 7 and 9 hours The results of the dissolution test are provided in FIG. 2. As can be seen, the dissolution rate was zero order for the capsule.

### Example 5

### Preparation of 250-mg Capsule for Pulsating Release of Mesalamine

72.5% 5-aminosalicylic acid was mixed with 22.5% HPMC K100 in a shear mixer. The mixture was then granulated using 3.9% povidone K30 dissolved in isopropyl alcohol. The granulated mixture was passed through a no. 10 mesh and allowed to air dry. When the granulated mixture was dry, it was then passed through a no. 20 mesh. The mixture was next lubricated with 1.3% magnesium stearate. The final mixture was mixed for 3 minutes. The mixture was loaded into a tableting machine.

A series of caplets was produced using 20 KN of force. Each caplet weighed 23.00 mg. Six of the caplets were coated using a film coating comprising 70% cellulose acetate phtalate as a film forming agent and 30% triethylcitrate as a plasticizer. At the same time, 9 caplets were coated using a film coating comprising 70% eudragit RS as a film forming agent and 30% dibutylphtalate as a plasticizer. The fifteen coated caplets were filled in a size 0 capsule to obtain a 250-mg mesalamine capsule.

### Example 6

### Preparation of 300-mg Capsule for Pulsating Release of Clindamycin Hydrochloride

87.0% clindamycin hydrochloride was mixed with 7.8% microcrystalline cellulose in a shear mixer. The mixture was then granulated using 3.9% povidone K30 dissolved in isopropyl alcohol. The granulated mixture was passed through a no. 10 mesh and allowed to air dry. When the granulated mixture was dry, it was then passed through a no. 20 mesh. The mixture was next lubricated with 1.3% magnesium stearate. The final mixture was mixed for 3 minutes. The mixture was loaded into a tableting machine.

A series of caplets was produced using 20 KN of force. Each caplet weighed 23.00 mg. Seven of the caplets were coated using a film coating comprising 70% cellulose acetate phtalate as a film forming agent and 30% triethylcitrate as a plasticizer. At the same time, eight caplets were coated using a film coating comprising 70% HPC as a film forming agent and 30% dibutylphtalate as a plasticizer. The fifteen coated caplets were filled in a size 0 capsule to obtain a 300-mg clindamycin hydrochloride capsule.

The inventive subject matter being thus described, it will be obvious that the same may be varied in many ways. Such variations are not to be regarded as a departure from the spirit and scope of the inventive subject matter, and all such modifications are intended to be included within the scope of the following claims.

1. An extended or controlled release encapsulated product, comprising:
a) at least one active ingredient;
b) at least one erodible polymer; and
c) at least one lubricating material; and
d) wherein said product is in the form of a caplet having a diameter from about 1 millimeter to about 7 millimeters and a length from about 1 millimeter to about 7 millimeters.

2. The extended or controlled release encapsulated product of claim 1 wherein said erodible polymer is a water soluble polymer.

3. The extended or controlled release encapsulated product of claim 2 wherein said water soluble polymer is selected from the group consisting of sodium carboxymethyl cellulose, hydroxyethyl cellulose, hydroxypropyl cellulose, propylene glycol alginate, sodium alginate, carboxymethyl cellulose and mixtures thereof.

4. The extended or controlled release encapsulated product of claim 1 wherein said erodible polymer is a water insoluble polymer.

5. The extended or controlled release encapsulated product of claim 4 wherein said water insoluble polymer is selected from the group consisting of cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, and mixtures thereof.

6. The extended or controlled release encapsulated product of claim 1 wherein said lubricating material is selected from the group consisting of: fats, emulsifiers, waxes, magnesium stearate, calcium stearate, talc, starches, silicon dioxide, and mixtures thereof.

7. The extended or controlled release encapsulated product of claim 1 wherein said diameter is about 3 millimeters and said length is about 3 millimeters.

8. The extended or controlled release encapsulated product of claim 1 wherein said product provides controlled release of said active ingredient.

9. The extended or controlled release encapsulated product of claim 1 wherein the product is coated with a polymeric material.

10. The extended or controlled release encapsulated product of claim 1 wherein the product is shaped in a manner to have a crown which has a thinner polymer coating than the rest of the caplet.

11. The extended or controlled release encapsulated product of claim 1 wherein said active ingredient is a pharmaceutical.

12. The encapsulated product of claim 11 wherein said pharmaceutical is selected from the group consisting of: antitussives, antihistamines, decongestants, alkaloids, mineral supplements, laxatives, antacids, ion exchange resins, anti-cholesterolemics, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psychotropics, antimanics, stimulants, gastrointestinal agents, sedatives, anti-diarrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs and mixtures thereof.

13. The encapsulated product of claim 12, wherein the said pharmaceutical is a psychotropic.

14. The encapsulated product of claim 13, wherein said psychotropic is a anti-anxiety therapeutic.

15. The encapsulated product of claim 13, wherein said psychotropic is an insomnia therapeutic.

16. The encapsulated product of claim 13, wherein said psychotropic is an antidepressant.

17. The encapsulated product of claim 16, wherein said antidepressant is selected from the group consisting of Fluoxetine HCl, Paroxetine HCl, Sertraline HCl, and Venlafaxine HCl, Amitriptyline, Nortriptyline, Imipramine, Desipramine, Doxepin, Trimipramine, Clomipramine, Protriptyline, Amoxapine, Maprotiline, Phenelzine, Tranylcypromine, Fluvoxamine, Venlafaxine, Trazodone, Nefazodone, Mirtazapine, Bupropion, or mixtures thereof.

18. The encapsulated product of claim 17, wherein said pharmaceutical is Fluoxetine HCl.

19. The encapsulated product of claim 12, wherein said pharmaceutical is a gastrointestinal therapeutic.

20. The encapsulated product of claim 19, wherein said gastrointestinal therapeutic is a ulcer therapeutic.

21. The encapsulated product of claim 20, wherein said ulcer therapeutic is selected from the group consisting of Omeprazole, Lansoprazole, Ranitidine HCl, Famotidine, Nizatidine, Teprenone, Cimetidine, Rabeprazole sodium, Sulpiride, or mixtures thereof.

22. The encapsulated product of claim 21, wherein said ulcer therapeutic is Omeprazole.

23. The encapsulated product of claim 19, wherein said gastrointestinal therapeutic is a anti-emetic.

24. The encapsulated product of claim 23, wherein said anti-emetic is selected from the group consisting of Ondansetron HCl, Granisetron HCl, dimenhydrinate, Tropisetron, Dolasetron mesylate, Cisapride, Sulfasalazine, Balsalazide, Infliximab, or mixtures thereof.

25. The encapsulated product of claim 24, wherein said anti-emetic is dimenhydrinate.

26. The encapsulated product of claim 19, wherein said gastrointestinal therapeutic is a anti-diarrheal therapeutic.

27. The encapsulated product of claim 26, wherein said anti-diarrheal therapeutic is selected from the group consisting of Loperamide HCl, diphenoxylate, codeine phosphate, camphorated opium tincture, or mixtures thereof.

28. The encapsulated product of claim 27, wherein said anti-diarrheal therapeutic is Loperamide HCl

29. The encapsulated product of claim 12, wherein said pharmaceutical is a migraine therapeutic.

30. The encapsulated product of claim 29, wherein said migraine therapeutic is selected from the group consisting of sumatriptan succinate, amitripyline, methysergide, propranolol, valproate, verapamil, dihydroergotamine, ergotamine, metoclopramide, naratriptan, prochlorperazine, rizatriptan benzoate, zolmitriptan, eletriptan, acetaminophen, aspirin, NSAID's, opioids, or mixtures thereof.

31. The encapsulated product of claim 30, wherein said migraine therapeutic is sumatriptan succinate.

32. The encapsulated product of claim 12, wherein said pharmaceutical is a therapeutic for the treatment of hypertension.

33. The encapsulated product of claim 32, wherein said therapeutic is selected from the group consisting of nifedipine, amlodipine besylate, losartan potassium, lisinopril, felodipine, benazepril HCl, ramipril, irbesartan, verapamil HCl, bisoprolol fumarate and hydrochlorothiazide, amlodipine and benazepril HCl, clonidine, candesartan, cilexetil, diltiazem, nicardipine, imidapril, trandolapril, eprosartan mesylate, nilvadipine, verapamil HCl, temocapril, prazosin HCl, isradipine, cilazapril, celiprolol, bisoprolol, betazolol HCl, ramipril, nisoldipine, lisinopril, trandolapril, and nisoldipine.

34. The encapsulated product of claim 33, wherein said therapeutic is nifedipine.

35. A pulsating release encapsulated product, comprising:
a) at least one active ingredient;
b) at least two erodible polymers, each of said erodible polymers having a different rate of dissolution or dissolving at a different pH; and
c) at least one lubricating material; and
d) wherein said product is in the form of a caplet having a diameter from about 1 millimeter to about 7 millimeters and a length from about 1 millimeter to about 7 millimeters.

36. The pulsating release encapsulated product of claim 35 wherein said erodible polymer is a water soluble polymer.

37. The pulsating release encapsulated product of claim 36 wherein said water soluble polymer is selected from the group consisting of sodium carboxymethyl cellulose, hydroxyethyl cellulose, hydroxypropyl cellulose, propylene glycol alginate, sodium alginate, carboxymethyl cellulose and mixtures thereof.

38. The pulsating release encapsulated product of claim 35 wherein said erodible polymer is a water insoluble polymer.

39. The pulsating release encapsulated product of claim 38 wherein said water insoluble polymer is selected from the group consisting of cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, and mixtures thereof.

40. The pulsating release encapsulated product of claim 35 wherein said lubricating material is selected from the group consisting of: fats, emulsifiers, waxes, magnesium stearate, calcium stearate, talc, starches, silicon dioxide, and mixtures thereof.

41. The pulsating release encapsulated product of claim 35 wherein said diameter is about 3 millimeters and said length is about 3 millimeters.

42. The pulsating release encapsulated product of claim 35 wherein said product provides controlled release of said active ingredient.

43. The pulsating release encapsulated product of claim 35 wherein the product is coated with a polymeric material.

44. The pulsating release encapsulated product of claim 35 wherein the product is shaped in a manner to have a crown which has a thinner polymer coating than the rest of the caplet.

45. The pulsating release encapsulated product of claim 35 wherein said active ingredient is a pharmaceutical.

46. The pulsating release product of claim 45 wherein said pharmaceutical is selected from the group consisting of: antitussives, antihistamines, decongestants, alkaloids, mineral supplements, laxatives, antacids, ion exchange resins, anti-cholesterolemics, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psychotropics, antimanics, stimulants, gastrointestinal agents, sedatives, anti-diarrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs and mixtures thereof.

47. A pulsating release product, comprising a capsule having a plurality of caplets, said caplets comprising:
a) at least one active ingredient;
b) at least one erodible polymer;
c) at least one lubricating material; and
d) wherein said caplet has a diameter from about 1 millimeter to about 7 millimeters and a length from about 1 millimeter to about 7 millimeters; and
wherein at least one of said plurality of caplets is prepared from an erodible polymer having a first dissolution rate, and at least another of said plurality of caplets is prepared from another erodible polymer having a second dissolution rate, and said first dissolution rate is not equal to said second dissolution rate.
